(19) United States
(12) Patent Application Publication
(10) Pub. No.: US 2009/0149936 A1
(43) Pub. Date: Jun. 11, 2009

(54) REINFORCED DELIVERY CATHETER

(75) Inventor: David Christian Lentz, Bloomington, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/950,781

(22) Filed: Dec. 5, 2007

(51) Int. Cl. A61F 2/84; A61M 25/00

(52) U.S. Cl. 623/1.11; 604/525

(57) ABSTRACT

A catheter for delivering an interventional device to a target site within the body of a patient. The catheter comprises an elongated shaft having a plurality of lumens extending longitudinally therein. The shaft has a proximal portion defining at least two lumens, an intermediate portion having fewer lumens than the proximal portion, and a distal portion configured for carrying the interventional device. The proximal and distal portions each have a greater stiffness than the stiffness of the intermediate portion. A stiffening member is incorporated into the intermediate portion of the shaft. The stiffening member is sized and positioned along the shaft in a manner such that the stiffness of the intermediate portion is increased relative to the stiffness of the proximal portion and the distal portion.

### BACKGROUND

1. Technical Field

The present invention relates to a catheter for use in delivering a medical interventional device to a target site within the body of a patient. More particularly, the invention relates to a reinforced delivery catheter for delivering a stent to a target site within the vasculature of a patient.

2. Background Information.

In modern medicine, interventional devices are often percutaneously introduced into the body of a patient via a suitable delivery apparatus, and delivered to a target site within the body for a medical purpose. One common example of an interventional device is a stent. A stent is typically inserted into the lumen of a vessel or other bodily passageway to reinforce, repair, or otherwise provide support to maintain the patency of the lumen. For example, in cardiovascular surgery a stent may be placed in the coronary artery at a location where the artery is weakened, damaged or otherwise susceptible to collapse. The stent, once in place, reinforces that portion of the artery, thereby allowing normal blood flow through the vessel.

One form of stent which is particularly desirable for implantation in arteries and other body lumens is a cylindrical stent which is radially expandable upon implantation from a smaller first diameter to a larger second diameter. Radially expandable stents are typically loaded onto, or into, a delivery catheter, and fed internally through the arterial pathways of the patient until the unexpanded stent reaches the target site. Radially expandable stents are normally of two general types. One type, generally referred to as a “balloon-expandable” stent, is fitted in a compressed state over an uninflated balloon at the distal end portion of the delivery catheter. Once the catheter reaches the target site, the balloon is inflated by transmitting an inflation fluid through a lumen in the delivery catheter to the interior of the balloon. Upon inflation, the balloon exerts a radial pressure on the stent, thereby causing the compressed stent to radially expand to a larger diameter. Following expansion, the stent exhibits sufficient radial rigidity to remain in the expanded condition after the balloon has been deflated and the catheter has been removed.

The other type of radially expandable stent, generally referred to as a “self-expanding” stent, is formed from a resilient or shape memory material which is capable of self-expanding from a compressed state to an expanded state without the application of a radial outwardly-exerted force on the stent. Typically, a self-expanding stent is loaded into a delivery device that restrains the stent in the compressed state. Once the delivery device is directed to the target site, an ejection mechanism, such as a pusher, is utilized to eject the stent from the distal end of the delivery device. Alternatively, an outer sheath of the delivery device is withdrawn such that it no longer covers the stent. In either event, once the stent is freed from the restraints of the device, it self-expands to the desired diameter.

The use of radially expandable stents advantageously allows the physician to insert relatively smaller diameter medical devices to reinforce or otherwise support relatively larger diameter vessels. However, the delivery of such stents to the target site has at times proven to be problematic. For example, the structure of a conventional delivery catheter may cause the catheter shaft to be subject to stress risers upon insertion of the shaft into a vessel. This can be particularly troublesome in delivery catheters of the type used to support balloon-expandable stents. Stress risers comprise weakened segments of the catheter which may cause the catheter shaft to undesirably bend, kink, or otherwise fail during insertion. Self-expanding stents, on the other hand, generally require the inclusion of an outer sheath or like structure to hold the stent in its compressed condition, and may require a pusher mechanism to force the stent out of the sheath at the site of expansion. The addition of an outer sheath adds bulk to the introducer apparatus, and increases its diameter. Any increase in the diameter of the introducer apparatus is inherently undesirable because it limits the size of the body vessel into which the apparatus can be introduced. Additionally, the necessity to include an outer sheath and pusher mechanism, along with the necessity to form the stent from the requisite expandable material, may add an undesirable level of complexity and cost to the assembly.

It is desired to provide a catheter for a self-expandable stent or other interventional medical device that avoids the problems of prior art devices.

### SUMMARY

The problems of the prior art are addressed by the features of the present invention. In one form thereof, the invention comprises a catheter for delivering an interventional device to a target site within the body of a patient. The catheter comprises an elongated shaft having a plurality of lumens extending longitudinally therein. The shaft has a proximal length defining at least two lumens, an intermediate length defining fewer lumens than the proximal length, and a distal length. The distal length is configured for carrying the interventional device. A stiffening member is incorporated into or onto at least a portion of the intermediate length of the shaft. The stiffening member is positioned along the shaft to increase the stiffness of the intermediate length portion relative to the stiffness of at least one of the proximal length and the distal length.

In another form thereof, the invention comprises a catheter for delivering a radially expandable interventional device to a target site within the body of a patient. The catheter includes an elongated shaft having a first length, a second length, and a third length. The first length extends from the proximal end toward the distal end of the shaft. The second length is distal to the first length, and the third length is distal to the second length. The shaft includes first and second lumens. An inflatable balloon spans at least a portion of the second and third lengths. The balloon is configured for carrying the interventional device in a radially compressed condition along at least the third length when the balloon is uninflated, and for radially expanding the interventional device when the balloon is inflated. The first lumen extends from the proximal end along the first length. A distal open end of the first lumen communicates with an interior of the balloon. The second lumen extends from the proximal end to the distal end along the respective first, second and third lengths. A stiffening member is embedded in the shaft along the second length.

In yet another form thereof, the invention comprises a catheter comprising an elongated shaft and a stiffening member. The elongated shaft has a proximal length, an intermediate length, and a distal length. The proximal length extends from the proximal end toward the distal end of the shaft. The intermediate length is distal to the proximal length, and the distal length is distal to the intermediate length. The intermediate length has a stiffness less than a stiffness of the proximal and distal lengths. The stiffening member is positioned at the shaft intermediate length. The stiffening member is sized and positioned along the shaft to increase the stiffness of the intermediate length relative to the stiffness of the proximal length and the distal length.

### BRIEF DESCRIPTION OF THE DRAWINGS

FIG. 1 is a side view of the distal portion of a prior art delivery catheter for a balloon expandable stent;

FIG. 2 is a side view of the distal portion of a delivery catheter according to an embodiment of the present invention;

FIG. 3 is an enlarged view of a portion of the shaft of the delivery catheter of FIG. 2;

FIG. 4 is a sectional view of the shaft taken along lines 4-4 of FIG. 3; and

FIG. 5 is a sectional view of the shaft portion of the delivery catheter, taken along lines 5-5 of FIG. 3.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the delivery catheter, as well as the axial ends of various component features. The term “proximal” is used in its conventional sense to refer to the end of the catheter (or component thereof) that is closest to the operator during use of the catheter. The term “distal” is used in its conventional sense to refer to the end of the catheter (or component thereof) that is initially inserted into the patient, or that is closest to the patient during use.

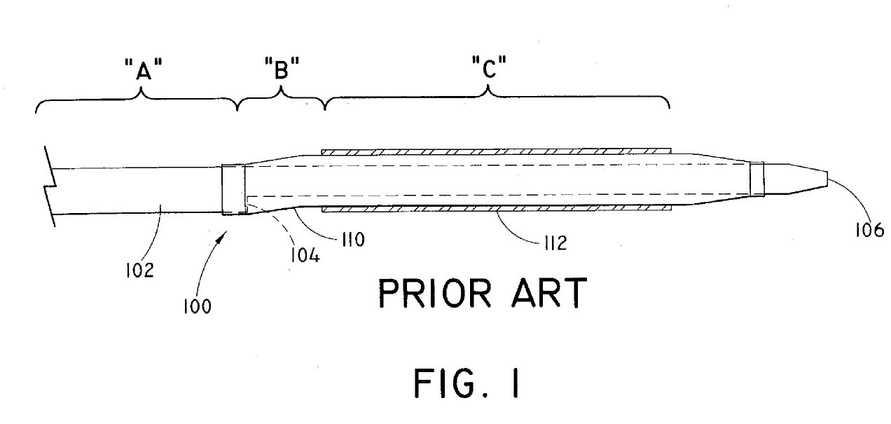

FIG. 1 illustrates a distal portion of a prior art catheter 100 of a type used for delivering a medical interventional device, such as a balloon expandable stent 112, to a target site within the body of a patient. In the embodiment shown, delivery catheter 100 comprises a catheter shaft 102, and a balloon 110 disposed at a distal end portion of the shaft. Balloon 110 is shown in its uninflated condition in FIG. 1. The medical interventional device, in this case stent 112, is shown crimped or otherwise compressed over the uninflated balloon in well-known fashion.

Shaft 102 of prior art catheter 100 includes dual lumens (not shown) extending longitudinally therethrough. One of the lumens is configured for carrying an inflation fluid from an inflation source to the interior of balloon 110. This lumen terminates at a point 104 proximal to the distal end of shaft 102, and communicates with the interior space of balloon 110. The other lumen serves as a conduit for a wire guide. This lumen extends longitudinally through the entire length of the shaft to distal end 106. The proximal portion of delivery catheter 100 is conventional, and need not be further shown and described to gain an understanding of the present invention.

The structure of the distal portion of conventional delivery catheter 100 illustrated in FIG. 1 includes three discrete lengths having differing stiffnesses. Length “A” comprises the length of the catheter shaft having dual lumens, and that terminates at point 104 along the shaft. Length “C” comprises the length of the catheter having a single lumen, and having the compressed stent 112 loaded thereon. Due to the presence of these structural and/or associated features (i.e., dual lumens, or single lumen having a stent loaded therein), each of lengths “A” and “C” has a relatively high stiffness. Length “B” is positioned axially intermediate lengths “A” and ” C”. Length “B” represents the length of catheter shaft 102 that has a single lumen, and that does not include additional stiffening structure, such as the stent that overlies length “C”. As a result, length “B” has a relatively low stiffness compared to the stiffness of respective lengths “A” and “C”. The presence of a length (“B”) having a relatively low stiffness between respective lengths (“A” and “C”) having relatively high stiffnesses may result in a stress riser being present at length “B”. A stress riser may cause the shaft to be subjected to increased risk of bending or failure along this length during use.

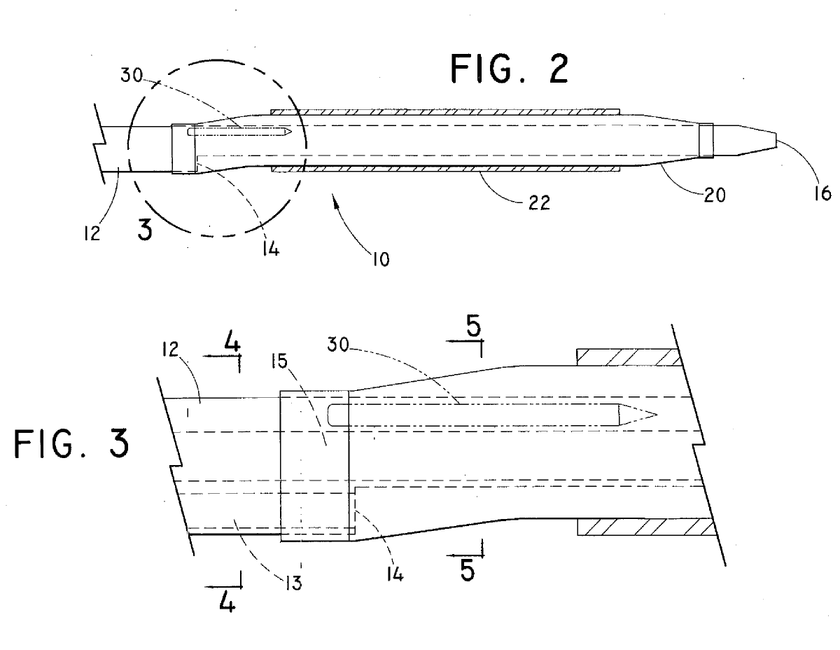

FIG. 2 illustrates a distal portion of a delivery catheter 10, according to an embodiment of the present invention. Delivery catheter 10 includes a catheter shaft 12, and a balloon 20 disposed at a distal end portion of shaft 12. Balloon 20 is shown in its uninflated condition in FIG. 2. A medical interventional device, in this case stent 22, is crimped or otherwise compressed over the uninflated balloon. Shaft 12 comprises at least two lumens extending therein. One of the lumens carries an inflation fluid from an inflation source (not shown) to the interior of balloon 20. This lumen terminates at a point 14 proximal to the distal end of shaft 12, and communicates with the interior of balloon 20 in well-known fashion. The other lumen extends longitudinally through the length of the shaft to distal end 16, and serves, e.g., as a conduit for a wire guide. The portions of delivery catheter 10 described above are generally similar to the corresponding features in the prior art catheter illustrated in FIG. 1. As with FIG. 1, the proximal portion of delivery catheter 10 is conventional, and need not be further shown and described to gain an understanding of the present invention.

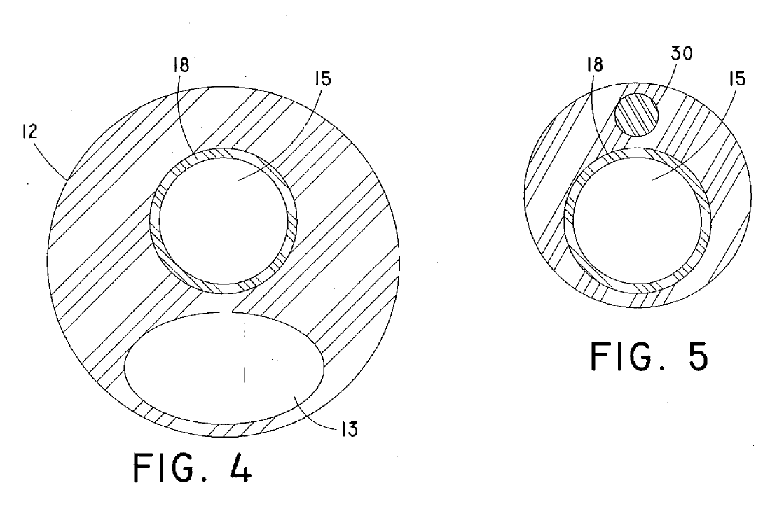

FIG. 3 is an enlarged view of a portion of the shaft 12 of the delivery catheter 10 of FIG. 2. FIG. 4 is a sectional view of the shaft taken along lines 4-4 of FIG. 3, and FIG. 5 is a sectional view of the shaft taken along lines 5-5 of FIG. 3. The balloon has been removed from the view shown in FIG. 5. FIG. 4 illustrates the dual lumens 13, 15 of the catheter shaft 12, and FIG. 5 illustrates lumen 15. FIG. 3 illustrates lumens 13, 15 in phantom. In the embodiment of FIGS. 3-5, lumen 13 is the inflation lumen, and lumen 15 is the wire guide lumen. Preferably, at least one of the lumens, in this case wire guide lumen 15, is lined with an inner layer 18 of a lubricious polymer, such as PTFE, in well-known fashion. In the preferred embodiment shown, wire guide lumen 15 has a generally circular cross section, and has a diameter sufficient to accommodate a wire guide. Inflation lumen 13 preferably has a more oval cross section. The relative size, shape, and orientation of the lumens shown in the figures is exemplary only, and those skilled in the art can readily fashion appropriate dimensions for the lumens for use in a particular sheath.

As illustrated in FIG. 3, catheter shaft 12 also includes a stiffening member, such as the stiffening rod 30 shown in phantom. The stiffening member is incorporated into the shaft of the delivery catheter to add strength to a portion of the shaft that is otherwise of low stiffness, and that is subject to increased risk of bending or failure. As a result, the stiffness of the shaft length reinforced by the stiffening rod is increased, relative to the stiffness of the shaft length portions not reinforced by the stiffening rod. Since the proximal and distal lengths are of higher stiffness than the intermediate length, the insertion of a stiffening rod along a discrete length (in this case the intermediate length) of the shaft can increase the stiffness of the intermediate length in a manner such that it at least approximates the stiffness of the proximal and/or distal length. As a result, the stress riser previously present in the less stiff portion (e.g., length “B” in FIG. 1) has been minimized, and can be eliminated entirely if desired, by selection of a stiffening member of an appropriate size and stiffness.

In the preferred embodiment shown, stiffening rod 30 comprises a wire or other generally elongated member that is embedded within the body of shaft 12. The rod preferably has a length such that it spans at least a portion of the shaft extending from lumen terminal point 14 to the proximal end of the stent. (FIG. 2). The length spanned by rod 30 may generally correspond to the low-stiffness length “B” of shaft 102 of prior art delivery catheter 100. (FIG. 1). Preferably, the rod will extend at least all of the length between lumen terminal point 14 and the proximal end of the stent. More preferably, rod 30 will extend an additional minor length (such as about 0.25 inch [6.35 mm]) in one, or both, axial directions beyond the lumen terminal point and the stent proximal end, to better ensure that no weakened lengths remain.

Preferably, the stiffening member, such as stiffening rod 30, is formed from a biocompatible metal, metal alloy, multi-filar material, or composite material. The stiffening member should be formed from a composition that is capable of providing sufficient strength to enhance the stiffness of a length of a catheter, such as length “B” in catheter 100, in a manner such that the stiffness of the reinforced length at least approximates that of one or more adjoining lengths, such as one or both of lengths “A” and “C” of catheter 100. Non-limiting examples of particularly suitable stiffening rod compositions include stainless steel and shape memory compositions such as nitinol. These compositions are widely used in medical devices, and a skilled artisan can readily craft an appropriate stiffening member from such compositions.

In most cases, it is expected that the stiffening member will have a diameter between about 0.001 and 0.006 inch [0.0254 and 0.152 mm], although other diameters may also be appropriate for a particular case. If desired, the rod can be tapered in the distal direction. The preferred diameter in any particular case will depend, of course, upon the diameter of the shaft. The stiffening member may have any cross-sectional profile, including round, flat, oval, etc. In order to minimize the profile of the stiffening member, a flat wire configuration will typically be preferred. Those skilled in the art will appreciate that the compositions and dimensions described herein are exemplary only, and that other compositions and dimensions may be substituted in an appropriate case to achieve enhanced stiffness as described herein.

FIG. 5 illustrates a sectional view of a portion of shaft 12 spanned by stiffening rod 30. In the preferred embodiment shown, stiffening rod 30 comprises an elongated structure that is embedded in an outer layer of shaft 12. By embedding stiffening rod 30 in shaft 12, and spanning the length between segments of higher stiffness as described, the shaft can be constructed such that no portion of the length of shaft 12 has a low stiffness, such as that described in length “B” of the prior art device. As a result, the reinforced length is not readily subject to inadvertent bending, kinking, etc., in the manner of the prior art device.

Delivery catheters are well known in the art, and shaft 12 can be formed of virtually any composition commonly utilized for such purposes. In a preferred embodiment, shaft 12 may comprise a layered structure comprising an inner liner of a lubricious polymer, such as polytetrafluoroethylene (PTFE), and an outer layer of a polymeric material, such as a polyether block amide (PEBA) or nylon. Preferably, the outer layer is a heat shrinkable tubular material, and the stiffening rod is embedded in the heat shrinkable (e.g., PEBA) layer by melting the layer around the rod in a manner such that the rod is embedded therein as shown in the figures. Techniques of embedding a member, such as stiffening rod 30, in a heat shrinkable tubular material are known in the art, and it is believed that virtually any such technique can be used to embed stiffening rod 30 in a fashion to provide reinforcement as described. Other suitable methods of incorporating, inserting, adhering, or otherwise retaining a stiffening member in a designated length of a shaft may be substituted.

The shaft can have a single durometer along its length, or it may have sections of varying durometers, typically aligned in descending fashion from a high durometer (e.g., high stiffness) at its proximal end to a low durometer (low stiffness) at its distal end, in well-known fashion. In addition, the shaft can include one or more radiopaque markers positioned along its length in well-known fashion.

Although illustrated herein in connection with a dual lumen shaft having side-by-side lumens, the use of a stiffening member as described herein is not limited to shafts having this configuration. For example, the shaft may have more than two lumens, and may be structured such that at least one of the lumens has a terminal point along its length (e.g., lumen terminal point 14), proximal to a terminal point of at least one other lumen or any other more distal segment of the shaft. In addition to the foregoing, a stiffening member may be used in similar fashion in connection with a coaxial dual lumen shaft, or any other multiple lumen shaft construction.

Furthermore, the use of a stiffening member as described herein is not limited to use with multi-lumen delivery catheters as described. Rather, a stiffening member may also be used to enhance the stiffness of a designated segment of any catheter or medical device that includes segments having relative stiffnesses that differ along the length of the device. Such segments will often be found at a portion of the device wherein a change of construction occurs, such as the lumen terminal point 14 described in the non-limiting example hereinabove. In devices having such segments of different stiffnesses, the stiffening member may simply be inserted, embedded, adhered, or otherwise added to the device along all, or at least some, of the length of a segment of lesser stiffness.

It is therefore intended that the foregoing detailed description be regarded as illustrative rather than limiting, and that it be understood that it is the following claims, including all equivalents, that are intended to define the spirit and scope of this invention.

1. A catheter for delivering an interventional device to a target site within the body of a patient, comprising:
an elongated shaft having a plurality of lumens extending longitudinally therein, said shaft having a proximal length defining at least two lumens, an intermediate length defining fewer lumens than said proximal length, and a distal length, said distal length configured for carrying said interventional device; and
a stiffening member incorporated at at least a portion of said intermediate length of said shaft, said stiffening member positioned along said shaft to increase a stiffness of said intermediate length portion relative to a stiffness of at least one of said proximal length and said distal length.

2. The catheter of claim 1, wherein said stiffening member is embedded in said intermediate length portion.

3. The catheter of claim 2, wherein said intermediate length is disposed generally adjacent said proximal length, and said distal length is disposed generally adjacent said intermediate length.

4. The catheter of claim 3, wherein the stiffening member is disposed along the entire intermediate length of the shaft.

5. The catheter of claim 3, wherein the stiffening member extends partially into at least one of the proximal and distal lengths of the shaft.

6. The catheter of claim 1, wherein the proximal length of the elongated shaft defines two lumens, and the intermediate length defines a single lumen.

7. The catheter of claim 1, wherein said interventional device comprises a stent, and at least a portion of the intermediate length and the distal length of said elongated shaft carries an inflatable balloon configured for carrying said stent in an unexpanded condition, said shaft lumens comprising an inflation lumen extending along said proximal length to said intermediate length, said inflation lumen communicating with an interior space of said balloon.

8. The catheter of claim 2, wherein said shaft comprises a heat shrinkable material, and said stiffening member is embedded in said heat shrinkable material.

9. The catheter of claim 8, wherein said heat shrinkable material comprises a polyether block amide, and said shaft further comprises an inner layer of a lubricious polymer.

10. A catheter for delivering a radially expandable interventional device to a target site within the body of a patient, comprising:
an elongated shaft having a proximal end and a distal end, said elongated shaft having a first length, a second length, and a third length, said first length extending from said proximal end toward said distal end, said second length distal to said first length, and said third length distal to said second length, said shaft comprising first and second lumens;
an inflatable balloon spanning at least a portion of said second and third lengths, said balloon configured for carrying said interventional device in a radially compressed condition along at least said third length when said balloon is uninflated, and for radially expanding said interventional device when said balloon is inflated;
said first lumen extending from said proximal end along said first length, a distal open end of said first lumen communicating with an interior of said balloon; and said second lumen extending from said proximal end to said distal end along said respective first, second and third lengths; and
a stiffening member embedded in said shaft along said second length.

11. The catheter of claim 10, wherein said stiffening member is positioned along said shaft in a manner to increase a stiffness of said second length relative to a stiffness of at least one of said first and third lengths.

12. The catheter of claim 11, wherein said stiffening member is embedded in said second length, and said stiffening member is positioned to increase the stiffness of said second length relative to the stiffness of each of said first and third lengths.

13. The catheter of claim 12, wherein the stiffening member spans the entire second length of the shaft, and extends partially into at least one of the first and third lengths of the shaft.

14. The catheter of claim 12, wherein said shaft comprises a heat shrinkable material, and said stiffening member comprises an elongated rod embedded in said heat shrinkable material.

15. The catheter of claim 14, wherein said heat shrinkable material comprises a polyether block amide, and said shaft further comprises an inner layer of a lubricious polymer.

16. The catheter of claim 12, wherein said stiffening member comprises a metal or metal alloy.

17. The catheter of claim 12, wherein said stiffening member comprises an elongated rod having a proximal end and a distal end, said rod tapering toward said distal end.

18. A catheter, comprising:
an elongated shaft having a proximal end and a distal end, said elongated shaft having a proximal length, an intermediate length, and a distal length, said proximal length extending from said proximal end toward said distal end, said intermediate length distal to said proximal length, and said distal length distal to said intermediate length, said intermediate length having a stiffness less than a stiffness of said proximal and distal lengths; and
a stiffening member positioned at said intermediate length, said stiffening member sized and positioned along said shaft to increase a stiffness of said intermediate length relative to a stiffness of said proximal length and said distal length.

19. The catheter of claim 18, wherein said stiffening member comprises a rod incorporated into said shaft along said intermediate length.

20. The catheter of claim 19, wherein said shaft comprises a heat shrinkable material, and said rod is embedded in said heat shrinkable material.
